(19) United States
(12) Patent Application Publication
(10) Pub. No.: US 2026/0168898 A1
(43) Pub. Date: Jun. 18, 2026

(54) DEVICE FOR STAINING BIOLOGICAL SAMPLES ADHERING TO A SPECIMEN SLIDE

(71) Applicant: BIO-GRAM DIAGNOSTICS GMBH, Worms (DE)

(72) Inventors: Petrit BLAKAJ, Heddesheim (DE); Ian Sebastian KLUTH, Mühltal (DE)

(21) Appl. No.: 19/128,616

(22) PCT Filed: Nov. 9, 2023
(86) PCT No.: PCT/EP2023/081289

§ 371 Date: May 9, 2025

(30) Foreign Application Priority Data
Nov. 11, 2022 (DE) 10 2022 129 863.9

(51) Int. Cl. G01N 1/44; G01N 1/28; G01N 1/30; G01N 1/31; G01N 1/36; G01N 35/00; G16H 40/60

(52) U.S. Cl. CPC G01N 1/44; G01N 1/2813; G01N 1/30; G01N 1/31; G01N 1/312; G01N 1/36; G01N 35/00029; G16H 40/60

(57) ABSTRACT

A device for staining biological samples adhering to a specimen slide, comprising at least one staining chamber with an opening for receiving a specimen slide in a fixed position, at least one staining agent container, at least one staining agent line, at least one staining agent pump and a control device wherein, by means of the at least one staining agent pump, staining agent is conveyable out of the at least one staining agent container through the at least one staining agent line into the at least one staining chamber.

The invention relates to a device for staining biological samples adhering to a specimen slide, comprising at least one staining chamber, at least one staining agent container, at least one staining agent line, at least one staining agent pump and a control device.

Such a device is known from U.S. Pat. No. 6,387,326 B1 . In the field of histology, i.e., the examination of biological tissue, staining methods are used to better distinguish tissue structures during an optical examination. Various staining methods are known in this context, for example HE staining (hematoxylin-eosin staining). With this staining, for example, cell nuclei of the tissue to be examined appear blue-purple and cytoplasm pink. Intracellular substances also stain, for example cartilage purple or fibers pink. Staining is also used in the field of hematology, i.e., the examination of blood and bone marrow.

The biological samples to be examined are placed on a specimen slide and then stained with different staining agents or staining methods depending on the examination method. In order to achieve optimum staining, it is important that the methods intended for staining, in particular the staining duration and the quantity of staining agent applied, are adhered to exactly. For this purpose, devices are known from the state of the art by means of which specimen slides with biological samples adhering to them can be automatically stained. In doing so, the specimen slides are usually transported through the device and subjected to a staining procedure.

Due to the fact that the specimen slides are transported through the device, it is possible to automatically treat a plurality of specimen slides one after the other. The dis-advantage is that the devices are complex and expensive due to the transport mechanism and the staining process adapted to the transport. Furthermore, the use of such devices only makes sense for larger quantities of specimen slides to be stained; for smaller quantities, the preparation and post-processing of the device, for example cleaning and rinsing, would be too great in relation to the stained samples.

The object of the invention is to provide a device for staining biological samples adhering to a specimen slide, which enables a smaller number of specimen slides to be stained while at the same time allowing flexible adaptation of the staining method.

This object is achieved with the features of claim 1. the dependent claims refer to advantageous embodiments.

The device according to the invention for staining biological samples adhering to a specimen slide comprises at least one staining chamber with an opening for receiving a specimen slide in a fixed position, at least one staining agent container, at least one staining agent line, at least one staining agent pump and a control device, wherein, by means of the staining agent pump, staining agents are conveyable out of the staining agent container via the staining agent line into the staining chamber.

In the device according to the invention, it is advantageous that the specimen slide remains in place in the staining chamber within the device and is subjected to the staining procedure there. For staining, the staining agent stored in a staining agent container is conveyed into the staining chamber via a staining agent pump, wherein the specimen slide arranged in the staining chamber is wetted with the staining agent. The device does not require any moving elements for transporting specimen slides and is therefore designed in a particularly simple and cost-effective manner. Accordingly, the device is particularly suitable for staining a small number of specimen slides.

In addition to the staining agent container, the device can also have a rinsing agent container and a collecting container. After staining is complete, a rinsing agent from the rinsing agent container can be applied to the specimen slide to rinse the specimen slide and remove excess staining agent. The rinsing solution, composed of staining agent and rinsing liquid, can be collected in the collecting container.

The staining chamber can be accommodated in a staining chamber housing, wherein the opening is also introduced in the staining chamber housing. Apart from the opening, the staining chamber housing is preferably closed on all sides and only has inlet openings and outlet openings for staining agent and rinsing solution in addition to the opening. The staining chamber is designed in a compact manner and has a relatively small volume, and allows an overall space-saving embodiment of the device.

The staining chamber housing can have a plurality of staining chambers, which are separated from each other by partition walls. The staining chambers can be arranged next to and/or on top of each other. Each staining chamber is designed to receive one specimen slide, so that each specimen slide can be stained separately in a staining chamber. This makes it possible in principle to stain a plurality of specimen slides simultaneously, wherein different staining methods can be carried out simultaneously in order to stain specimen slides as desired.

The opening can be formed as a slit. The width and height of the slit is based on the dimensions of the specimen slide to be stained and is preferably matched to the specimen slide. As a result, the area of the opening is small and the interior of the staining chamber is largely separated from the surroundings, in particular preventing the staining agent or rinsing solution in the staining chamber from drying out. Furthermore, odor nuisance or health hazards caused by liquids evaporating in the device, such as staining reagents or rinsing solutions, can be reduced.

The circumferential edge of the opening can be associated with a seal. Said seal is preferably formed in such a way that it rests tightly against a specimen slide arranged in the staining chamber. This can further reduce odor nuisance and/or health hazards caused by reagents leaking from the device. The seal can be designed as a lip seal, wherein the lip seals against the specimen slide on all sides when the specimen slide is inserted.

At least one inlet opening and at least one outlet opening can be inserted into the staining chamber, wherein the inlet opening is connected to the staining agent line.

Staining agent is inserted into the staining chamber via the inlet opening, where it wets the specimen slide that is arranged in the staining chamber. The staining agent can flow out of the staining chamber via the outlet opening. Once staining is complete, the specimen slides can be rinsed with a rinsing solution. The rinsing solution can be inserted via the inlet opening or a further inlet opening can be provided for the supply of rinsing solution. The rinsing solution flows out through the outlet opening.

The staining chamber housing can have a plate-shaped bottom part which is connected to a plate-shaped top part, wherein at least one recess is introduced in the bottom part and/or the top part for the embodiment of the staining chamber. Such a staining chamber housing is easy and cost-effective to manufacture. Preferably, the staining chamber housing is formed of metallic material.

A seal, for example a gasket, can be arranged between the bottom part and the top part. The inlet opening and/or the outlet opening preferably open into the bottom part. To stain the specimen slides, said slides are arranged in the staining chamber such that the biological samples adhering to the slide are facing the bottom part. In doing so, the specimen slide can rest flat on the bottom part or in the recess that is inserted in the bottom part. When a staining agent is inserted into the staining chamber via the inlet opening, the staining agent wets the specimen slide automatically over the entire surface of said slide due to the capillary effect, wherein excess staining agent flows out via the outlet opening.

The staining chamber housing can be arranged inclined in the device. The staining chamber housing is preferably arranged in such a way that the staining chambers are inclined to the horizontal. In doing so, the end of the staining chamber facing the opening is higher than the end of the staining chamber opposite the opening. On the one hand, this prevents staining agent and/or rinsing solution from escaping outward through the opening. On the other hand, a particularly good distribution of the staining agent over the surface of the specimen slide is achieved, especially if the inlet opening is located in the area of the opening and the outlet opening is associated with the end of the staining chamber opposite the opening.

The outlet opening can be connected to a suction pump, wherein the suction pump can be connected to the outlet opening via an outlet line. Staining agent and/or rinsing solution can be actively removed from the staining chamber via the suction pump. The control device is preferably configured to influence the staining agent pump in order to convey staining agent from the staining agent container into the staining chamber. Thus, the control device influences the staining agent pump to stain the biological sample adhering to the specimen slide.

The staining chamber can have a contact area for supporting the specimen slide and a depression delimited by the contact area. The contact area is preferably arranged circumferentially in the edge region of the staining chamber and enables a specimen slide to be supported on its edge region. The depression is preferably formed bowl shaped and the bottom of the depression is spaced from a specimen slide placed on the contact area. The depression can be designed in such a way that a capillary effect is formed in the depression, by means of which liquids, such as staining agents and rinsing solutions, are automatically distributed in the depression and on the surface of the specimen slide. However, it is also conceivable that liquids are actively pumped into and out of the depression.

The at least one inlet opening and the at least one outlet opening can be arranged in the depression. This allows liquids to come into contact with the specimen slide particularly easy and quick.

By means of the suction pump, a vacuum is generatable in the depression, due to which vacuum the specimen slide is fixable in the staining chamber. This fixes the specimen slide in the staining chamber. Liquids can wet the specimen slide and, in doing so, do not leak from the staining chamber in the process.

The staining agent pump can be configured to generate a pulsed staining agent flow. This can be achieved in particular with peristaltic pumps, which usually have a pulsed flow due to their function. The pulsed flow ensures particularly fast and thorough wet-ting of the specimen slide, so that an improved staining result is achieved, and processing times are shortened. The effect of the pulsed flow is particularly good if there is a vacuum in the depression when the specimen slide is in place and the pulsed flow enters the depression and is guided out of it again. A further improvement is achieved if the depression is designed in such a way that liquids are also distributed in said depression by capillary effect.

The inlet opening and the outlet opening can be fitted with quick-release fasteners, which allow the hoses of the staining agent line and outlet line to be changed quickly and without tools.

Staining agent types and/or staining agent quantities and/or staining agent exposure times adapted to different biological samples and/or different evaluation methods can be stored in the control device. For this purpose, the control device preferably comprises a memory in which various evaluation methods and staining methods as well as the associated procedures for the staining are stored. In particular, it is conceivable to store procedures for the following staining methods in the control device:
Hematoxylin-eosin staining (for histology),
Pappenheim staining (for hematology),
Wright-Giemsa staining (for cytology),
Gram staining (for bacterial analysis),
Ziehl-Neelsen staining (for tuberculosis analysis), and
Papanicolau staining (for gynecology).

Different staining agents and rinsing solutions are used for the different staining methods and the exposure time of the staining agents and the need for rinsing and/or drying differs for the different staining methods. The different procedures for the different staining methods are stored or storable for this purpose in the control device, so that switching to a different staining method is particularly easy. Moreover, it is conceivable to modify individual process parameters of a staining procedure, for example the quantity of staining agent and/or rinsing agent conveyed to the inlet openings or even a possible temperature control of the staining chamber. This allows individual parameters to be adjusted to achieve better staining results.

The staining chamber can be temperature-controlled. For this purpose, the bottom part and/or the top part can be equipped with a heating element. Alternatively, it is also conceivable to arrange a heating element between the bottom part and the top part. The heating element is preferably formed as an electric resistance heater. Temperature control can speed up staining processes, particularly because drying times can be shortened. Process times can be almost doubled by increasing the process temperature by just 20° C. Another advantage is that temperature control can ensure particularly consistent process conditions.

The control device can be configured to record staining methods that have been carried out and store them. This makes it possible to trace the method and process parameters used to stain the specimen slides afterwards. In particular, it is also conceivable that stored stainings are output to an external storage medium via an interface.

An input device is preferably associated with the control device. Preferably, the input device is designed as a touch display. The input device makes it particularly easy to select staining agent methods and modify the process parameters associated with individual methods. Furthermore, the control device can have an interface via which alternative staining procedures can be read into the control device.

Some embodiments of the device according to the invention are explained in more detail below with reference to the figures. These show, each schematically:

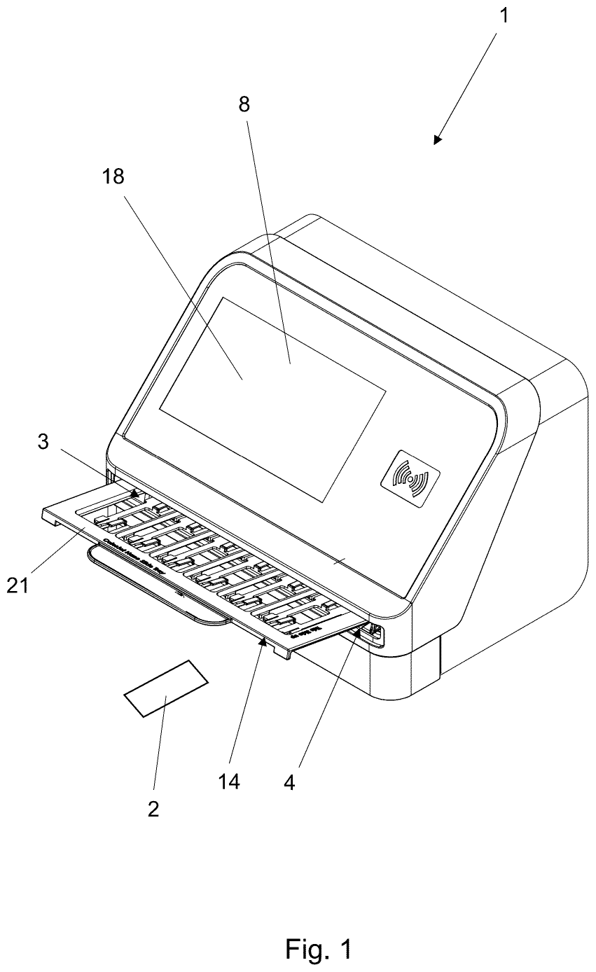

FIG. 1 a device with an open slide-in device;

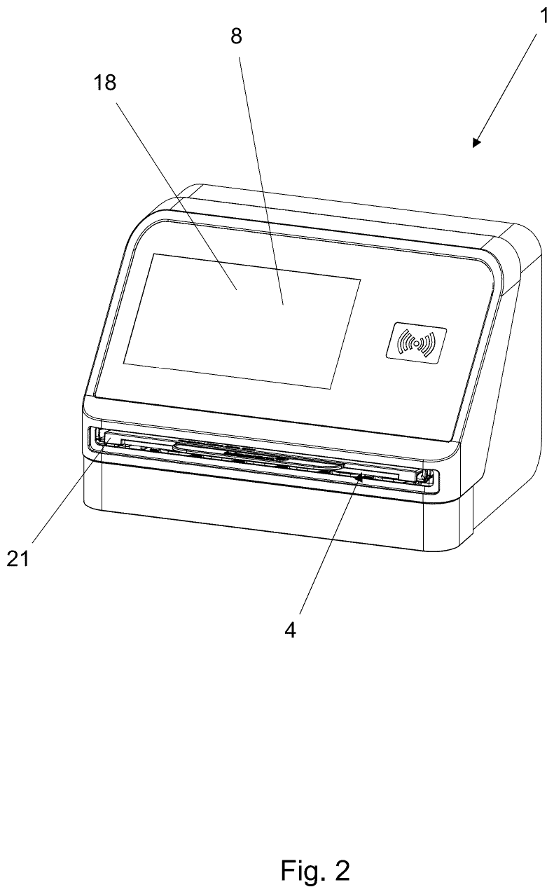

FIG. 2 the device according to FIG. 1 with a closed slide-in device;

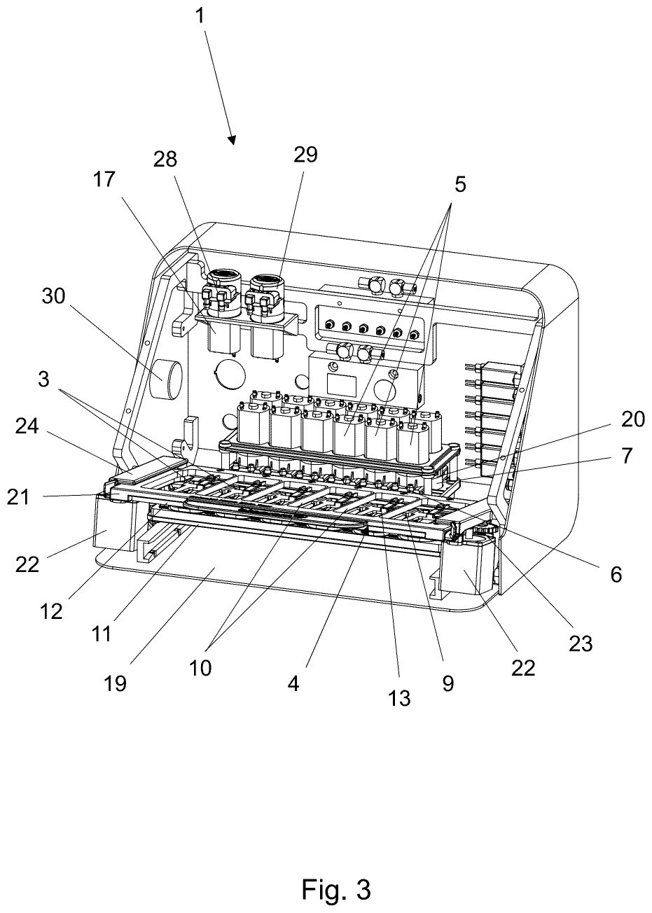

FIG. 3 the device according to FIG. 1 and FIG. 2 open, without input device;

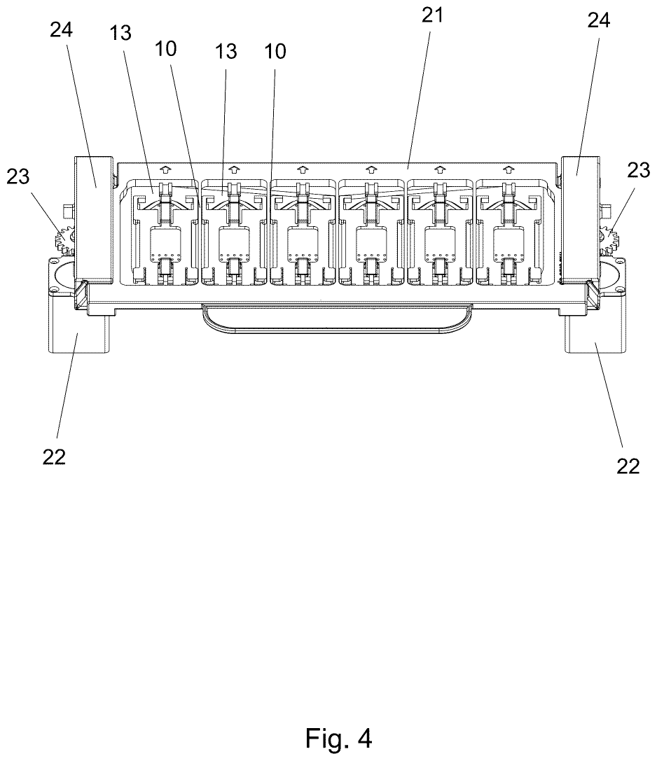

FIG. 4 a detailed top view of the slide-in device;

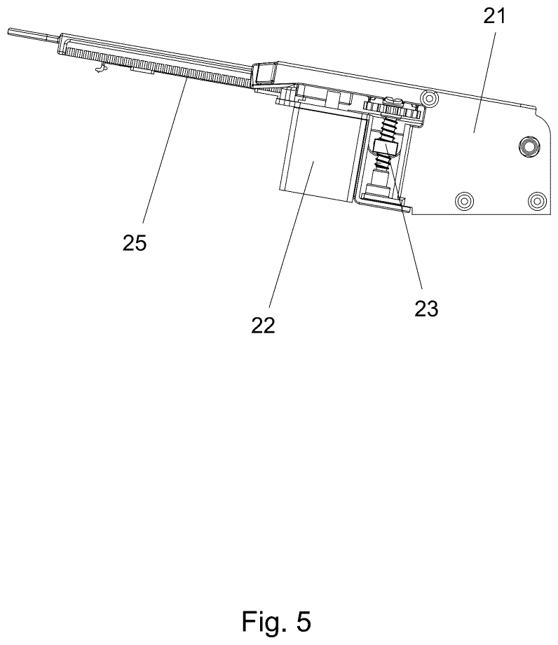

FIG. 5 the slide-in device in side view;

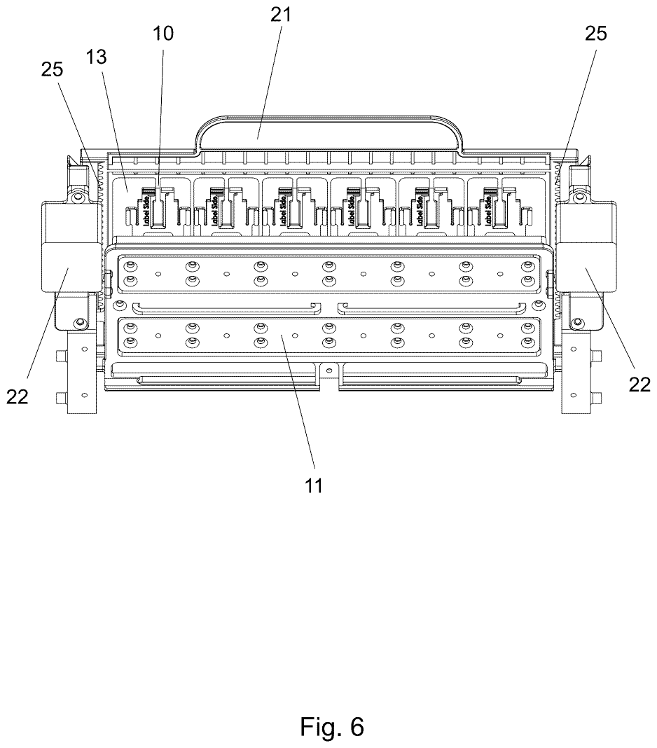

FIG. 6 in detail, the staining chamber housing of the device in bottom view;

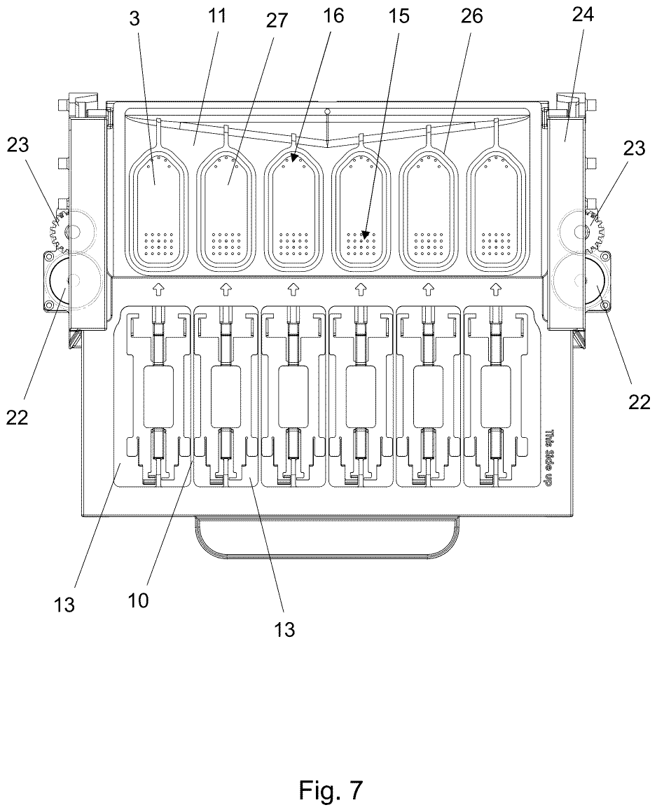

FIG. 7 the staining chamber housing in top view;

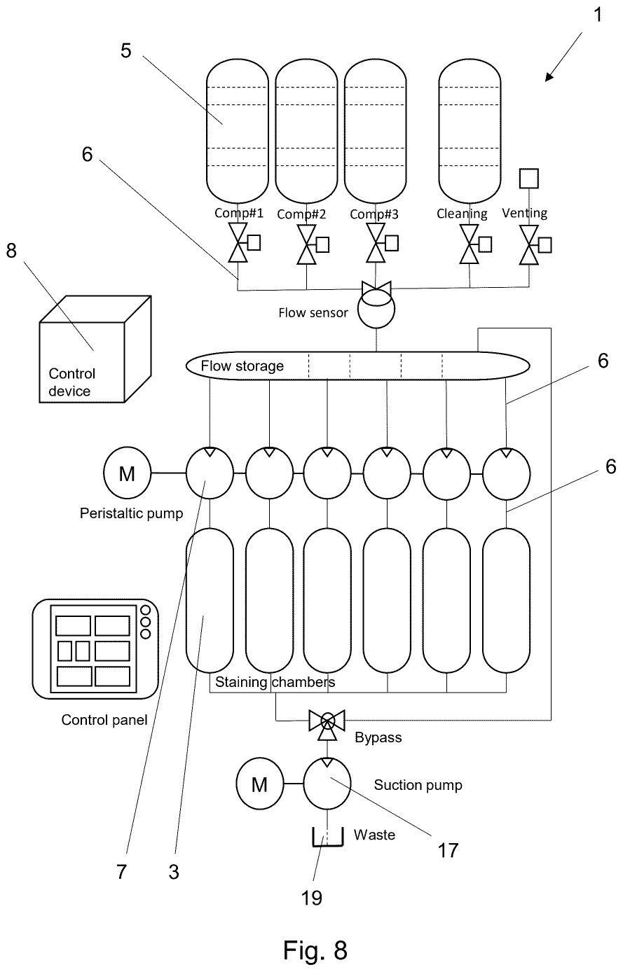

FIG. 8 a process diagram.

FIG. 1 shows a device 1 for staining biological samples adhering to a specimen slide 2. The biological samples adhering to the specimen slide 2 are treated in the device 1 with one or more staining agents in order to stain the structures of the biological samples differently so that they can be subsequently better distinguished during an optical examination, for example during microscopy.

The device 1 comprises a plurality of staining chambers 3, each of which is provided with an opening 4 for receiving a specimen slide 2 in a fixed position. The staining chambers 3 are accommodated in a staining chamber housing 9, wherein the openings 4 are introduced in the staining chamber housing 9. In the present embodiment, the staining chamber housing 9 has a plurality of staining chambers 3 arranged next to each other, which are separated from each other by partition walls 10. The openings 4 are each designed as a slit, wherein the cross-sectional area of the slits essentially correspond to the cross-section of the specimen slides 2 to be examined. Each opening 4 is provided with a seal 14 in the form of a lip seal, wherein the seal 14 is associated with the circumferential edge of an opening 4 in each case and projects inwards, so that the seal 14 is in sealing contact with the specimen slide 2 on all sides when the specimen slide 2 is inserted.

In the present embodiment, a slide-in device 21 in the form of a drawer is associated with the staining chambers 3. FIG. 1 shows the slide-in device 21 in the open position. FIG. 2 shows the device 1 according to FIG. 1 with inserted, closed slide-in device 21.

The slide-in device 21 has receiving sections in which a plurality of specimen slides 2 can be placed at the same time. By inserting the slide-in device 21 into the device 1, all specimen slides 2 are guided simultaneously into a separate staining chamber 3. Insertion of the slide-in device 21 is performed by an electric motor via the stepper motors 22 connected to the gear 23 of the slide-in device. The slide-in device 21 is provided with seals 14, which ensure the sealing of the staining chambers 3 to each other and the sealing of the staining chambers 3 in the direction of the opening 4.

The device 1 further comprises a plurality of staining agent containers 5, staining agent lines 6 and a plurality of staining agent pumps 7 as well as a control device 8.

The control device 8 is configured to influence the staining agent pumps 7 in order to stain the biological samples adhering to the specimen slide 2. The control device 8 stores staining agent types, staining agent quantities and staining agent exposure times adapted to different biological samples and/or different evaluation methods. The device 1 is configured to perform the following stainings:
Hematoxylin-eosin staining (for histology),
Pappenheim staining (for hematology),
Wright-Giemsa staining (for cytology),
Gram staining (for bacterial analysis),
Ziehl-Neelsen staining (for tuberculosis analysis), and
Papanicolau staining (for gynecology).

An input device 18 in the form of a touch display is associated with the control device 8. The control device 8 is also equipped with a memory for storing the staining procedures. The memory can also be used to save stainings that have been carried out. The stored stainings can also be output via an interface and transferred to an external storage medium, for example. In the control device 8, predefined procedures and process parameters are stored for the staining methods described above. The process parameters can be modified via the input device and the modification can also be saved in the control device 8. This makes it possible to change predetermined procedures, for example to change staining results or to adapt the device 1 to changed staining agents. Adapted parameters can also be output via the interface.

FIG. 3 shows the device 1 according to FIG. 1, wherein in this illustration the unit forming a cover consisting of control device 8 and input device 18 is removed, so that the view into the interior of the device 1 is unobstructed. The unit is positively fixed in the device 1 and can be removed without tools for maintenance purposes. This is particularly advantageous because the lines 6 formed of flexible hoses are subject to wear and must be replaced regularly for reasons of hygiene alone.

In the present embodiment, the device 1 comprises as many staining agent pumps 7 as staining chambers 3, so that each staining chamber 3 is assigned with a staining agent pump 7. The staining agent pumps 7 convey staining agent out of the staining agent containers 5 via the staining agent lines 6 into the staining chambers 3. The staining agent pumps 7 are designed as peristaltic pumps, wherein the peristaltic pumps are modular and a plurality of staining agent pumps 7 are coupled together. In this embodiment, each staining agent pump 7 is assigned with an electric motor 20 in the form of a stepper motor. In an alternative embodiment, the staining agent pumps 7 are coupled in such a way that they are controlled by a single electric motor 20. The staining agent pumps 7 are configured to generate a pulsed staining agent flow.

The staining chamber housing 9 is arranged inclined in the device 1; the angle of inclination to the horizontal is ten degrees. The inclination is designed in such a way that the staining chamber housing 9 is lowered towards the rear wall of the device 1.

The staining agent removed after the staining process is fed to a liquid reservoir 28. A rinsing agent container 29 for receiving rinsing solution is arranged adjacent to the liquid reservoir 28.

A scanner 30 is arranged on one side wall of the device 1. In the present embodiment, the scanner 30 is equipped to read QR codes and is used to read QR codes that are attached to the staining agent containers 5. The information read in is for-warded to the control device 8. By scanning the staining agent containers 5 immediately before assembly, operating errors can be avoided, and the scanned information can be used for program selection and quality assurance.

FIG. 4 shows a detailed top view of the slide-in device 21. In this illustration, in particular the laterally arranged stepper motors 22 along with the gear 23 can be seen.

The slide-in device 21 is mounted in laterally arranged guide rails 24.

FIG. 5 shows a side view of the slide-in device 21 according to FIG. 4. In this illustration, the toothed racks 25 can be seen, which are attached to the side of the slide-in device, and which are in operative connection with the gear 23 in order to be able to move the slide-in device 21 relative to the staining chamber housing 9.

FIG. 6 shows in detail the bottom view of the staining chamber housing 9 with the staining chambers 3 arranged therein and the slide-in device 21. In this illustration, in particular the inlet openings 15 and outlet openings 16 opening into the bottom part 11 and the lines connected to them can be seen.

The staining chamber housing 9 comprises a plate-shaped bottom part 11, which is connected to a plate-shaped top part 12. In the present embodiment, the recesses 13 forming the staining chambers 3 are introduced in the bottom part 11. The bottom part 11 and top part 12 are formed of metallic material and are firmly connected to each other via a screw connection.

Inlet openings 15 and outlet openings 16 are introduced in each of the staining chambers 3, wherein the inlet openings 15 are connected to the staining agent line 6. The outlet openings 16 are connected to a collecting container 19. A suction pump 17 is arranged between the outlet openings 16 and the collecting container 19.

FIG. 7 shows a top view of the staining chamber housing 9 according to FIG. 6. In this illustration, it is particularly easy to see that depressions 27 are inserted in the bottom part 11, which are delimited by circumferential contact areas 26. The inlet openings 15 and the outlet openings 16 are arranged in the depressions, wherein the inlet openings are associated with one narrow side and the outlet openings 16 with the other narrow side. By means of the suction pump 17, a vaccum can be generated in the depression 27 when specimen slide 2 is placed on it, by means of which the specimen slide 2 is fixed in the staining chamber 3 with a force fit.

FIG. 8 shows a process diagram and a schematic representation of the components of device 1 described above.

What is claimed is:

1. A device for staining biological samples adhering to a specimen slide, the device comprising:
at least one staining chamber with an opening for receiving a specimen slide in a fixed position;
at least one staining agent container;
at least one staining agent line;
at least one staining agent pump; and
a control device,
wherein, by means of the at least one staining agent pump, staining agent is conveyable out of the at least one staining agent container through the at least one staining agent line into the at least one staining chamber.

2. The device according to claim 1, wherein the at least one staining chamber is accommodated in a staining chamber housing, and wherein the opening is introduced in the staining chamber housing

3. The device according claim 2, wherein the at least one staining chamber includes a plurality of staining chambers, and wherein the staining chamber housing includes the plurality of staining chambers which are separated from one another by partition walls.

4. The device according to claim 3, wherein a staining agent pump of the at least one staining agent pump is assigned to each staining chamber of the plurality of staining chambers.

5. The device according to claim 1, wherein the opening is formed as a slit.

6. The device according to claim 1, wherein a seal is assigned to the circumferential edge of the opening.

7. The device according to claim 1, wherein at least one inlet opening and at least one outlet opening are introduced in the at least one staining chamber, and wherein the inlet opening is connected to the at least one staining agent line.

8. The device according to claim 2, wherein the staining chamber housing has a plate-shaped bottom part which is connected to a plate-shaped top part, wherein at least one recess is introduced in the bottom part and/or the top part for the embodiment of the at least one staining chamber.

9. The device according to claim 2, wherein the staining chamber housing is arranged inclined in the device.

10. The device according claim 7, wherein the at least one outlet opening is connected to a suction pump.

11. The device according to claim 1, wherein the control device influences the at least one staining agent pump in order to convey staining agent out of the at least one staining agent container into the at least one staining agent chamber.

12. The device according to claim Z, wherein the at least one staining chamber has a contact area for supporting the specimen slide and a depression delimited by the contact area.

13. The device according to claim 12, wherein the at least one inlet opening and the at least one outlet opening are arranged in the depression.

14. The device according to claim 13, wherein a vacuum is generatable in the depression by the suction pump, by means of which the specimen slide is fixed in the at least one staining agent chamber.

15. The device according to claim 1, wherein the at least one staining agent pump is configured to generate a pulsed staining agent flow.
